(19) United States
(12) Patent Application Publication
(10) Pub. No.: US 2003/0091605 A1
(43) Pub. Date: May 15, 2003

(54) Use of alpha-lipoic acid for producing cosmetic or dermatological preparations for regenerating stressed skin, in particular aged skin

(75) Inventors: Christopher Mummert, Bienenbuttel (DE); Claudia Mundt, Bremen (DE); Thomas Blatt, Hamburg (DE); Inge Kruse, Hamburg (DE); Franz Stab, Echem (DE); Uwe Schonrock, Nahe (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/092,126

(22) Filed: Mar. 6, 2002

(30) Foreign Application Priority Data
Mar. 6, 2001 (DE) 101 11 048.0

(51) Int. Cl. A61K 31/385; A61K 7/00

(52) U.S. Cl. 424/401; 514/440

(57) ABSTRACT

Use of α-lipoic acid for producing cosmetic or dermatological preparations for regenerating stressed skin, in particular aged skin.

The present invention relates to the use of α-lipoic acid for producing cosmetic or dermatological preparations for regenerating stressed skin, in particular aged skin.

Cosmetic skin care is primarily understood as meaning strengthening or restoring the natural function of the skin as a barrier against environmental influences (for example, dirt, chemicals, microorganisms) and against the loss of endogenous substances (for example, water, natural fats, electrolytes).

If this function is disturbed, it will come to an increased resorption of toxic or allergenic substances, or an attack of microorganisms, and consequently to toxic or allergenic skin reactions.

For example, in the case of aged skin, a regenerative restoration occurs in a slowed-down manner. In this process, the capacity of the horny layer to bind water diminishes in particular. For this reason, the skin becomes inflexible, dry, and cracked (“physiologically” dry skin). The consequence is a barrier damage. The skin becomes susceptible to negative environmental influences, such as the invasion of microorganisms, toxins, and allergens, possibly resulting in toxic or allergenic skin reactions.

In the case of pathologically dry and sensitive skin, barrier damage exists a priori. Epidermal intercellular lipids become deficient or develop in an inadequate quantity or composition. The consequence is an increased permeability of the horny layer and an inadequate protection of the skin against loss of hygroscopic substances and water.

The barrier effect of the skin may be quantified by way of determining the transepidermal water loss (TEWL). This process involves the evaporation of water from the interior of the body without including the loss of water during perspiration. The determination of the TEWL value has shown to be extremely informative, and may be used for diagnosing cracked or chapped skin, for determining the compatibility of chemically differently composed surfactants and the like.

For the beauty and well-cared appearance of the skin, the portion of water in the uppermost skin layer is of greatest importance. It is possible to influence the water portion in a favorable manner, to a limited extent, by introducing moisture regulators.

Anionic surfactants, which are in general ingredients of cleansing preparations, are capable of increasing the pH value in the horny layer long-lastingly, which greatly impedes regenerative processes that would otherwise serve to restore or renew the barrier function of the skin. In this instance, a new, often very unfavorable state of equilibrium develops in the horny layer between regeneration and loss of essential substances as a result of regular extraction. This state of equilibrium decisively affects the outer appearance of the skin and the physiological functioning of the horny layer.

A simple water bath alone without addition of surfactants causes an initial swelling of the horny layer of the skin, with the degree of this swelling being dependent on the duration of the bath and its temperature. At the same time, not only water-soluble substances, such as, for example, water-soluble dirt particles, but also skin-inherent substances, which are responsible for the capacity of the horny layer to bind water, are rinsed off or washed away. In addition, skin-inherent, surface-active substances also cause skin fat to be separated and washed away to a certain extent. This causes after an initial swelling a subsequent, clear drying of the skin, which may still be increased by wash-active additives.

In the case of a healthy skin, these processes are in general meaningless, since the protective mechanisms of the skin are capable of compensating such slight disturbances of the upper skin layers without any problems. However, already in the case of nonpathological deviations from a normal state, for example, by environmentally caused damage from wear, or irritations, light damage, aged skin, etc., the protective mechanism of the skin surface is disrupted. Under circumstances, same will then be no longer capable of fulfilling its task out of its own forces, and must be regenerated by external measures.

Moreover, it is known that lipid composition and lipid quantity of the horny layer of the pathologically changed, dry, and the dry but not yet diseased skin of younger and older people deviate from the normal condition, which is found in the healthy, normally hydrated skin of a same age group. In this connection, changes in the lipid patterns of the very dry, non-eczematous skin of patients with an atopic eczema represent an extreme case for the deviations, which are found in the dry skin of humans with a healthy skin.

These deviations relate quite particularly to ceramides, which are greatly reduced in their quantity and, in addition, differently composed. In this connection, the deficit of ceramides 1 and 3 comes as a very special surprise, it being known in particular in the case of ceramide 1 that it increases in a special way the order of the lipids in the intercellular membrane systems.

Disadvantageous changes in the lipid membranes of the aforesaid kind are possibly based on a faultily controlled lipid biosynthesis, and in the end effect they likewise increase the transepidermal water loss. A long-lasting barrier weakness in turn makes a skin that is per se healthy, more sensitive, and may contribute in the individual case to the development of eczematous processes in the diseased skin.

Normally, the effect of ointments and creams on the barrier function and hydration does not consist of a restoration or strengthening of the physico-chemical properties of the lamellae from intercellular lipids. A substantial partial effect is based on the mere covering of the treated skin regions and on the resultant water collection in the subjacent horny layer. Co-applied hygroscopic substances bind the water, so that a measurable increase of the water content in the horny layer develops. However, it is relatively easy to remove again this merely physical barrier. After discontinuing the product, the skin will then return very rapidly to its condition before the start of the treatment. Moreover, the effect of skin care in the case of regular treatment may slow down, so that finally the status quo is again reached even during the treatment. In the case of certain products, after discontinuing their use, the condition of the skin deteriorates, under circumstances temporarily. Thus, a long-lasting effect of the product is normally not achieved, or only to a limited extent.

To assist the deficient skin in its natural regeneration, and to strengthen its physiological function, it has lately become more and more common to add topical preparations to the mixtures of intercellular lipids, which are to be used by the skin for rebuilding its natural barrier. However, these lipids, in particular the ceramides, are very expensive raw materials. In addition, their effect is mostly much less than hoped for.

It is therefore an object of the present invention to find ways of avoiding the disadvantages of the art. In particular, it is desired that the effect of the skin care products be physiologically fast and long-lasting.

Skin care in the meaning of the present invention means in first place that the natural function of the skin as a barrier against environmental influences (for example, dirt, chemicals, microorganisms) and against the loss of endogenous substances (for example, water, lipids, electrolytes) is strengthened or restored.

Products for the care, treatment, and cleansing of dry and stressed skin are known per se. However, their contribution to the regeneration of a physiologically intact, hydrated and smooth horny layer is limited in terms of scope and time.

The action of ointments and creams on the barrier function and the hydration of the horny layer is based substantially on the coverage (occlusion) of treated skin regions. The ointment or creme represents so-to-speak a (second) artificial barrier, which is intended to prevent a loss of water. In a corresponding manner, this physical barrier is again easy to remove—for example, with cleansing agents—thereby reaching again the original, impaired condition. Moreover, the effect of the skin care may slow down in the case of regular treatment. After discontinuing the product application, the skin returns again very quickly to its condition before the start of the treatment. In the case of certain products, the condition of the skin even deteriorates under circumstances temporarily. Thus, a long-lasting effect of the product is not achieved, or only to a limited extent.

The effect of some pharmaceutical preparations on the barrier function of the skin even consists in a selective barrier damage, which is intended to make it possible that active substances are able to penetrate into the skin or through the skin into the body. In this connection, a disturbed appearance of the skin is accepted as a side effect with partial approval.

The effect of cleansing products for the care consists in essence in an efficient reverse lubrication with sebum lipid-like substances. As a result of simultaneously reducing the surfactant content of such preparations, it is possible to further limit the damage to the horny layer barrier.

However, the state of the art lacks of preparations, which positively influence the barrier function and the hydration of the horny layer, and strengthen or even restore the physico-chemical properties of the horny layer and in particular of the lamellae from intercellular lipids.

Thus, it was the object of the present invention to eliminate the disadvantages of the art. In particular, it is intended to make available preparations for the skin care and preparations for the cleansing the skin, which maintain or restore the barrier properties of the skin, particularly when the natural regeneration of the skin is not adequate. Furthermore, they are to be suited for the treatment and prophylaxis of consequent damage from the drying of the skin, for example, fissures or inflammatory or allergic processes, or even neurodermatitis. It was also an object of the invention to make available stable, skin-caring, cosmetic and/or dermatological agents, which protect the skin against environmental influences, such as sun and wind. In particular, it is desired that the effect of the preparation be physiological, fast, and long-lasting.

Furthermore, it is an object of skin care to compensate the loss of fat and water caused by daily washing. This will be very important in the instance, when the capacity of natural regeneration is inadequate. Furthermore, the skin care products are intended to protect against environmental influences, in particular sun and wind, and to delay aging of the skin.

Chronological aging of the skin is caused, for example, by endogenous, genetically determined factors. In the epidermis and dermis, aging causes, for example, the following structural damage and functional disturbances, which may also fall under the term “senile xerosis,” namely:

a) dryness, roughness, and formation of dryness wrinkles;

b) itching; and

c) reduced relubrication by sebaceous glands (for example, after washing).

Exogenous factors, such as UV light and chemical noxae, can have a cumulative effect, and accelerate or supplement, for example, endogenous aging processes. In the epidermis and dermis, exogenous factors cause in particular, for example, the following structural damage and functional disturbances in the skin, which go beyond the extent and quality of damage in the case of chronological aging:

d) visible vascular dilations (teleangiectasis, cuperosis;

e) flaccidity and formation of wrinkles;

f) local hyperpigmentation, hypopigmentation, abnormal pigmentation (for example, age spots); and

g) increased susceptibility to mechanical stress (for example, cracking).

The invention relates in particular to products for the care of the naturally aged skin, as well as for treating consequent damage from photoaging, in particular the phenomena listed under a) through g).

Products for the care of aged skin are known per se. They contain, for example, retinoids (vitamin A acid and/or derivatives thereof) or vitamin A and/or its derivatives thereof. Their effect on structural damage is however limited in terms of scope. Moreover, considerable difficulties exist in the product development with respect to stabilizing the active ingredients to an adequate extent against oxidative decay. The use of products that contain vitamin A acid, moreover, often causes erythematous skin irritations. Retinoids can therefore be used only in low concentrations.

In particular, the present invention relates to cosmetic preparations having not only an effective protection against harmful oxidation processes in the skin, but also for the protection of cosmetic preparations themselves or for the protection of the ingredients against harmful oxidation processes.

In a preferred embodiment, the present invention relates to cosmetic and dermatological preparations for the prophylaxis and treatment of cosmetic or dermatological skin changes, such as, for example, skin aging, in particular skin aging caused by oxidative processes.

In a further preferred embodiment, the present invention relates to combinations of active ingredients and preparations, which are used for the prophylaxis and treatment of light-sensitive skin, in particular photodermatoses.

The harmful effect of the ultraviolet portion of solar radiation on the skin is generally known. While rays of a wavelength smaller than 290 nm (the so-called UV range) are absorbed by the ozone layer in the earth's atmosphere, the rays in the range between 290 nm and 320 nm, the so-called UVB range, cause erythema, a simple sun burn, or even burns of greater or lesser severity.

A narrower range of about 308 nm is given as a maximum of the erythema activity.

Numerous combinations are known for protecting against UVB radiation. These are derivatives of 3-benzylidene camphor, 4-aminobenzoic acid, cinnamic acid, salicylic acid, benzophenone, as well as 2-phenyl benzimidazole.

Likewise, for the range from 320 nm to about 400 nm, the so-called UVA range, it is important to have available filter substances, since the rays thereof may cause reactions in the case of a light-sensitive skin. It has been found that UVA radiation leads to damage of the elastic and collagenous fibers of the connective tissue, which causes the skin to age prematurely, and that it is to be seen as the cause for numerous phototoxic and photoallergic reactions. The harmful influence of the UVB radiation can be intensified by the UVA radiation.

As a result, certain derivatives of dibenzomethane are used for the protection against the rays of the UVA range, the photostability of which is not given to an adequate extent (Int. J. Cosm. Science 10, 53 (1988).

However, UV radiation can also lead to photochemical reactions, in which case photochemical reaction products intervene in the skin metabolism.

Such photochemical reaction products are predominantly radical compounds, for example hydroxyl radicals, singlet oxygen. Undefined radical photoproducts, which form in the skin itself, can also display uncontrolled consequent reactions because of their high reactivity. However, singlet oxygen, a non-radical excited state of the oxygen molecules, can also be formed during UV radiation, as can short-lived epoxides and many others. For example, singlet oxygen differs from the normally present triplet oxygen (radical ground state) by an increased reactivity. However, excited reactive (radical) triplet states of the oxygen molecule also exist.

Furthermore, UV radiation pertains to the ionizing radiation. This means that there is the risk that ionic species will also form in a UV exposure, which then for their part are able to intervene oxidatively in biochemical processes.

To prevent these reactions, it is possible to incorporate additional antioxidants and/or radical scavengers into the cosmetic or dermatological formulations.

It has already been suggested to use vitamin E, a substance with known antioxidative effect in light-protective formulations. However, even in this case, the achieved effect remains far behind the expected effect.

It was therefore also the object of the invention to create cosmetic, dermatological, and pharmaceutical active ingredients and preparations as well as light-protective formulations, which are used for the prophylaxis and treatment of light-sensitive skin, in particular photodermatoses, preferably PLD.

Other names for polymorphous light dermatosis include PLD, PLE, Mallorca acne, and a large number of additional names, such as are specified in the literature (for example, A. Voelkel et al., Zentralblatt Haut- und Geschlechtskrankheiten (1989), 186, p. 2).

Antioxidants are mainly used as protective substances against deterioration of the preparations, in which they are present. Nonetheless, it is known that undesired oxidation processes may also occur in the human and the animal skin. Such processes play an important role in the case of skin aging.

The essay “Skin Diseases Associated with oxidative Injury” in “Oxidative Stress in Dermatology, p. 323 et seq. (Marcel Decker Inc., New York, Basel Hong Kong, Editor: Jürgen Fuchs, Frankfurt, and Lester Packer, Berkeley, Calif.), describes oxidative injuries of the skin and their more likely causes.

Also for the reason of preventing such reactions, it is possible to incorporate additional antioxidants and/or radical scavengers in cosmetic or dermatological formulations.

Some antioxidants and radical scavengers are known. Thus, U.S. Pat. Nos. 4,144,325 and 4,248,861, as well as numerous other documents have already proposed to use vitamin E with its known antioxidant action in light-protective formulations. However, likewise in this instance, the achieved effect remains far behind the expected effect.

Thus, it was an object of the invention to find ways of avoiding the disadvantages of the art. In particular, it is desired to eliminate the effect of the injuries connected with endogenous, chronological, and exogenous skin aging, and that the prophylaxis be durable, long-lasting, and without the risk of side effects.

In accordance with the invention, the inconveniences of the art are eliminated by the use of α-lipoic acid for producing cosmetic or dermatological preparations for stressed skin, in particular aged skin.

Cosmetic or dermatological preparations that contain α-lipoic acid are in any respect quite satisfactory preparations. It was not foreseeable for the persons of skill in the art that preparations of the present invention would

better maintain or restore the barrier properties of stressed skin, in particular aged skin; better counteract drying of stressed skin, in particular aged skin;

better act against skin aging; and

better protect stressed skin, in particular aged skin against environmental influences than preparations of the art.

The use of α-lipoic acid and cosmetic or topical dermatological preparations with an effective content of α-lipoic acid enables in a surprising manner not only an effective treatment, but also a prophylaxis of

deficient, sensitive, or hypoactive skin conditions or deficient, sensitive, or hypoactive conditions of skin appendages;

phenomena of premature aging of the skin (for example, wrinkles, age spots, teleangiectases) and/or of skin appendages;

environmentally caused changes (smoking, smog, reactive oxygen species, free radicals) and in particular light-induced, negative changes of the skin and skin appendages;

light-induced skin damage;

pigmentation disorders;

itching;

dry skin conditions and barrier disorders of the horny layer;

hair loss and improved hair growth;

inflammatory skin conditions as well as atopical eczema, seborrheic eczema; polymorphous light dermatosis, psoriasis, and vitiligo.

However, α-lipoic acid and cosmetic or topical dermatological preparations with an effective content of α-lipoic acid are also useful in a surprising manner

for soothing sensitive or irritated skin;

for stimulating the synthesis of collagen, hyaluronic acid, and elastin;

for stimulating the ceramide synthesis of the skin;

for stimulating the intracellular DNA synthesis, in particular in the case of deficient or hypoactive skin conditions;

for increasing cell renewal and regeneration of the skin;

for increasing the skin's own protection and repair mechanisms (for example, for dysfunctional enzymes, DNA, lipids, and proteins); and

for pretreating and aftertreating in cases of topical application of laser- and abrasive treatments, which serve, for example, to reduce skin wrinkles and scars to counteract resulting skin irritations and to promote the regeneration processes in the damaged skin.

Accordingly it is also advantageous to use α-lipoic acid according to the invention in the case of dry skin that is found sensitive.

According to the invention it is also advantageous to use α-lipoic acid for producing cosmetic or dermatological preparations for the treatment and/or prophylaxis of the symptoms of intrinsic and/or extrinsic skin aging, as well as for the treatment and prophylaxis of the harmful effects of ultraviolet radiation on the skin.

In 1952, α-lipoic acid was isolated from liver tissue, and its structure was clarified as a sulfur-containing fatty acid. Bacteria, plants, and higher organisms are capable of producing themselves α-lipoic acid in their metabolism. The question of a natural biosynthesis remains still unanswered for humans.

α-lipioc acid is used for the therapy of polyneuropathy, a sensitivity disorder on hands and feet resulting from diabetes. 200 to 600 milligrams of α-lipoic acid a day lead to a significant reduction of the pain intensity. The energy metabolism of the hand and foot nerves is activated by α-lipoic acid, thereby resulting in a better nerve conductivity and, with that, in reduced numbness feeling and reflex failures.

α-lipoic acid lowers pathologically increased liver values and promotes healing of hepatitis. α-lipoic acid is contained in small amounts in most nutrients. Relatively high values are to be found only in meat. It is a recognized fact that α-lipoic acid possesses strongly antioxidant properties.

WO 97/10808 and U.S. Pat. No. 5,472,698 disclose the cosmetic use of α-lipoc acid against symptoms of skin aging. DE 42 42 876 discloses combinations of active substances from biotin and antioxidants with α-lipoic acid for the cosmetic and/or dermatological care of the skin and/or skin appendages, as well as cosmetic and/or dermatological preparations that contain such combinations of active ingredients.

The preparations of the present invention advantageously comprise 0.001 to 10 wt. % α-lipoic acid based on the total weight of the preparations.

According to the invention, common antioxidants may be used in preparations, which contain α-lipoic acid.

Advantageously, such antioxidants are selected from the group consisting of amino acids (for example, glycine, histidine, tyrosine, trytophan) and derivatives thereof; imidazoles (for example, urocanic acid) and their derivatives; peptides, such as D,L-carnosine, D-carnosine, L-carnosine, and derivatives thereof (for example, anserine); carotenoids, carotene (for example, α-carotene, β-carotene, lycopene) and derivatives thereof; aurothioglucose, propylethiouracil and other thiols (for example, thioredoxin, glutathione, cysteine, cystine, cystamine, and glycosyl-, N-acetyl-, methyl-, ethyl-, propyl-, amyl-, butyl- and lauryl-, palmitoyl-, oleyl-, γ-linoleyl-, cholesteryl-, and glyceryl esters thereof, as well as their salts, dilauryl thiodipropionate, distearyl hiodipropionate, thiodipropionic acid and derivatives thereof (esters, ethers, peptides, lipids, nucleotides, nucleosides, and salts), and sulfoximine compounds (for example, buthionine sulfoximines, homocysteine sulfoximine, buthionine sulfones, penta-, hexa-, heptathionine sulfoximine) in very low tolerated doses (for example, pmol to μmol/kg); furthermore metal chelating agents (for example, α-hydroxy fatty acids, palmitic acids, phytic acid, lactoferrin), α-hydroxy acids (for example, citric acid, lactic acid, malic acid), humic acid, bile acid, bile extracts, bilirubin, biliverdin, EDTA, EGTA and derivatives thereof; unsaturated fatty acids and derivatives thereof (for example, γ-linolenic acid, linoleic acid, oleic acid), folic acid, and derivatives thereof; alanine diacetic acid, flavonoids, polyphenoles, catechins, vitamin C and derivatives (for example, ascorbyl palmitate, Mg-ascorbyl phosphate, ascorbyl acetate); tocopherols and derivatives thereof (for example, vitamin E acetate); as well as coniferyl benzoate of the benzoic resin; rutinic acid and derivatives thereof; ferulic acid and derivatives thereof; butylhydroxytoluene; butylhydroxyanisole; nordihydroguaicaic resin acid; nordihydroguaiaretic acid; trihydroxybutyrophenone; uric acid and derivatives thereof, mannose and derivatives thereof; zinc and derivatives thereof (for examle, ZnO, ZnSO₄); selenium and derivatives thereof (for example, selenium methionine); stilbene and derivatives thereof (for example, stilbene oxide, trans-stilbene oxide); and derivatives (salts, esters, ethers, sugar, nucleotides, nucelosides, peptides, and lipids) of these referenced active ingredients, which are suitable according to the invention.

The amount of the antioxidants (one or more compounds) in the preparations is preferably from 0.001 to 30 wt.%, more preferably 0.05 to 20 wt. %, in particular 1 to 10 wt. % based on the total weight of the preparation.

Prophylaxis or cosmetic or dermatological treatment with α-lipoic acid, or with the cosmetic or topical dermatological preparations with an active content of α-lipoic acid occurs in the usual way, namely in such a manner that α-lipoic acid or the cosmetic or topical dermatological preparations with an active content of α-lipoic acid is applied to the affected places of the skin.

Advantageously, the α-lipoic acid may be included in common cosmetic and dermatological preparations, which can have a variety of forms. They can be in the form of, for example, a solution, an emulsion of the water-in-oil (W/O) type, or of the oil-in-water (O/W) type, or multiple emulsions of the water-in-oil-in water (W/O/W) type, or oil-in-water-in-oil (O/W/0) type, a hydrosdispersion or lipodispersion, a gel, or solid stick, or even an aerosol.

Emulsions according to inventions in the meaning of the present invention, for example, in the form of a cream, a lotion, a cosmetic milk, are advantageous and contain, for example, fats, oils, waxes, and/or other fatty matters, as well as water and one or more elmusifiers, as are normally used for such a type of formulation.

It is also possible and advantageous in the meaning of the present invention to add the active substance in use according to the invention to aqueous systems or surfactant preparations for cleansing the skin and hair.

Naturally, it is known to the person of skill in the art that in most cases, exacting cosmetic preparations are not possible without the usual adjuvants and additives. The cosmetic preparations according to the invention can therefore contain cosmetic adjuvants as are customarily used in such preparations, for example, preservatives, bactericides, deodorants, antiperspirants, insect repellents, vitamins, antifoaming agents, dyes, pigments with a coloring action, thickeners, emollients, moisturizers and/or humectants, fats, oils, waxes, or other common ingredients of a cosmetic formulation, such as alcohols, polyols, polymers, foam stabililzers, electrolytes, organic solvents, or silicone derivatives.

Mutatis mutandis, corresponding requirements apply to the formulation of medicinal preparations.

Medicated topical compositions in the meaning of the present invention normally contain one or more medicaments in active concentrations. For the sake of simplicity, the legal provisions of the Federal Republic of Germany (for example, statutory regulation for cosmetics, food and medicament law) are herewith incorporated by reference for purposes of making a clear distinction between cosmetic and medicinal application and corresponding products.

Advantageously, preparations of the present invention may additionally contain substances, which absorb UV radiation in the UVB range, where the total amount of the filter substances is, for example, from 0.1 wt. % to 30 wt. %, preferably 0.5 wt. % to 10 wt. %, in particular 1.0 to 6.0 wt. % based on the total weight of the preparations for making available cosmetic preparations, which protect hair and skin against the entire range of the ultraviolet radiation. They can also be used as sunscreen agents for the hair.

When the preparations of the present invention contain UVB filter substances, same may be oil-soluble or water-soluble. In accordance with the invention, advantageous oil-soluble UVB filters include, for example:

derivatives of 3-benzylidene camphor, preferably 3-(4-methylbenzylidene) camphor, 3-benylidene camphor;

derivatives of 4-aminobenzoic acid, preferably 2-(ethylhexyl)₄-dimethylamino-benzoate, amyl 4-(dimethylamino)-benzoate;

esters of cinnamic acid, preferably (2-ethylhexyl)₄-methoxy cinnamate, isopentyl 4-methoxy cinnamate;

esters of salicylic acid, preferably (2-ethylhexyl) salicylicate, (4-isopropylbenzyl) salicylicate, homomenthyl salicylicate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate; and

2,4,6-trianilino-(p-carbo-2′-ethyl-1′-hexyloxy)-1,3,5-triazine.

Advantageous water-soluble UVB filters are, for example:

salts of 2-phenylbenzimidazole-5-sulfonic acid, such as its sodium-, potassium-, or its triethanol ammonium salt, as well as the sulfonic acid itself;

sulfonic acid derivatives of benzophenones, preferably, 2-hydroxy-4-methoxybenopenone-5-sulfonic acid and its salts; and

sulfonic acid derivatives of 3-benzylidene camphor, such as, for example, 4-(2-oxo-3-bornylidene-methyl)benzenesulfonic acid, 2-methyl-S-(2-oxo-3-bornylidenemethyl)sulfonic acid and its salts, as well as the 1,4-di(2-oxo-10-sulfo-3-bornylidene)benzene and salts thereof (the corresponding 10-sulfato compounds, for example the corresponding sodium-, potassium-, or triethanolammonium salt), also referred to as benzene-1,4-di(2-oxo-3-bornylidenemethyl-lo-sulfonic acid.

Naturally, the list of the referenced UVB filters, which may be used in combination with a-lipoic acid according to the invention, is not intended to be limiting.

It may also be of advantage to use UVA filters, which are normally present in cosmetic preparations. These substances are preferably derivatives of dibenzoylmethane, in particular 1-(4′-tert-butylphenyl)-3-(4′-methoxyphenyl)-propane-1,3-dione and 1-phenyl-3-(4′-isopropylphenyl)propane-1,3-dione. The amounts used can be the same as the amounts for the UVB combination.

Advantageously, cosmetic and dermatological preparations in accordance with the invention contain in addition inorganic pigments based on metal oxides and/or other metal compounds that are difficult to dissolve in water or insoluble, in particular the oxides of titanium (TiO₂), zinc (ZnO), iron (for example, Fe₂O₃), zirconium (ZrO₂), silicon (SiO₂), manganese (for example, MnO), aluminum (Al₂O₃), cerium (for example (Ce₂O₃), mixed oxides of the corresponding metals, as well as mixtures of such oxides. Especially preferred are pigments on the basis of TiO₂.

While not required, it will be especially advantageous in the meaning of the present invention, when the inorganic pigments are present in hydrophobic form, i.e., they are treated for water-repellency on the surface. This surface treatment may consist in that the pigments are provided with a thin, hydrophobic layer by a method known per se.

One of such methods consists, for example, in producing the hydrophobic surface layer by a reaction according to

nTiO₂+m(RO₃)Si—R′→nTiO₂ (superficial),

where n and m are stoichiometric parameters that are to be inserted as desired, and R and R′ are the desired organic radicals. For example, pigments that are made hydrophobic analogously to DE-OS 33 14 742 are of advantage.

Advantageous TiO₂ pigments are commercially available under the trade names MT 100T from TAYCA™, furthermore M 160 from Kemira™, as well as T 805 from Degussa™.

Preparations according to the invention can also contain anionic, nonanionic, and/or amphoteric surfactants, in particular when crystalline or microcrystalline solids, for example, inorganic micropigments are to be included in the preparations of the present invention. Surfactants are amphophilic substances, which are capable of dissolving organic, nonpolar substances in water.

The hydrophilic moieties of a surfactant molecule are in most cases polar functional groups, for example —COO⁻, OSO₃²⁻, OSO₃⁻, whereas the hydrophobic parts normally represent nonpolar hydrocarbon residues. In general, surfactants are classified according to the type and charge of the hydrophilic moiety of the molecule. In this connection, it is possible to differentiate between four groups:

anionic surfactants;

cationic surfactants;

amphoteric surfactants; and

nonionic surfactants.

As functional groups, anionic surfactants normally include carboxylate-, sulfate-, and sulfonate groups. In an aqueous solution, they form negatively charged, organic ions in the acidic or neutral environment. Cationic surfactants are characterized nearly exclusively by the presence of a quaternary ammonium group. In an aqueous solution, they form positively charged, organic ions in the acidic or neutral environment. Amphoteric surfactants contain both anionic and cationic groups, and accordingly in an aqueous solution act as anionic or cationic surfactants depending on the pH value. In a strongly acidic environment, they possess a positive charge, and in an alkaline environment a negative charge. In the neutral pH range, however, they are zwitterionic, as is demonstrated in the following example:

| | | |
|---|---|---|
| RNH₂⁻₂CH₂COOH X⁻ | (at pH = 2) | X⁻ = any desired |
| | | anion, e.g., Cl⁻ |
| RNH₂⁺CH₂CH₂COO⁻ | (at pH = 7) | |
| RNHCH₂CH₂COO⁻ B⁺ | (at pH = 12) | B⁺ = any desired |
| | | cation, e.g., |
| | | Na⁺ |

Typical of nonionic surfactants are polyether chains. Nonionic surfactants form no ions in an aqueous medium.

### A. Anionic Surfactants

Anionic surfactants that can be advantageously used, include: acylamino acids (and salts thereof), such as:

1. Acylglutamate, for example, sodium acyl glutamate, di-TEA-palmitoyl asparate, and sodium caprylic/capric glutamate;

2. Acylpeptides, for example, palmitoyl-hydrolyzed milk protein, sodium cocoyl-hydrolyzed soy protein, and sodium/potassium cocoyl-hydrolyzed collagen;

3. Sarcosinates, for example, myristoyl sarcosinate, TEA-lauroyl sarcosinate, sodium lauroyl sarcosinate, and sodium cocoyl sarcosinate;

4. Taurates, for example, sodium lauroyl taurate and sodium methylcocoyl taurate;

5. Acyl lactylates, lauroyl lactylate, caproyl lactylate; and

6. Alaninates.

Carboxylic acids and derivatives, such as:

1. Carboxylic acids, for example, lauric acid, aluminum stearate, magnesium alkanolate, and zinc undecylenate;

2. Ester carboxylic acids, for example, calcium stearoyl lactyllate, laureth-6 citrate, and sodium PEG-4 lauramide carboxylate; and

3. Ether carboxylic acids, for example, sodium laureth-13 carboxylate, and sodium PEG-6 cocoamide carboxylate.

Esters of phosphoric acid and salts, such as, for example, DEA-oleth-10-phosphate and dilaureth-4 phosphates.

Sufonic acids and salts, such as:

1. Acyl isethionate, for example, sodium-ammoniumcocoyl isethionate;

2. Alkyaryl sulfonates;

3. Alkyl sulfonates, for example, sodium cocosmonoglyceride sulfate, sodium C₁₂₋₁₄ olefin sulfonate, sodium lauryl sulfoacetate, and magnesium PEG-3 cocamide sulfate;

4. Sulfosuccinates, for example, dioctyl sodium sulfosuccinate, disodium laureth sulfosuccinate, disodium lauryl sulfosuccinate, and disodium undecylenamido-MEA-sulfosuccinate;

as well as esters of sulfuric acid, such as:

1. Alkyl ether sulfonates, for example, sodium, ammonium, magnesium, MIPA, TIPA, laureth sulfate, sodium myreth sulfate, and sodium C₁₂₋₁₃ pareth sulfate; and

2. Alkyl sulfates, for example, sodium, ammonium, and TEA-lauryl sulfate.

### B. Cationic Surfactants

Cationic surfactants that can be advantageously used, include:

1. Alkylamines,

2. Alkylimidazoles,

3. Ethoxylated amines, and

4. Quaternary surfactants.

5. Esterquats

Quaternary surfactants contain at least one nitrogen atom, which is covalently bonded with 4 alkyl- or aryl groups. Irrespective of the pH value, this leads to a positive charge. Advantageous are alkylbetain, alkylamidopropylbetain, and alkyl-amidopropylhydroxysultain. The cationic surfactants that are used in accordance with the invention, can also be selected from the group of quaternary ammonium compounds, in particular benzyltrialkyl ammoniumchlorides or bromides, such as, for example, benzyldimethylstearyl ammonium chloride, furthermore alkyltrialkyl ammonium salts, for example, cetyltrimethyl ammonium chloride or bromide, alkyidimethylhydroxyethyl ammonium chloride or bromide, dialkyldimethyl ammonium chloride or bromide, alkylamidethyltrimethyl ammonium ether sulfates, alkylpyridinium salts, for example, lauryl or cetyl pyrimidinium chloride, imidazoline derivatives and compounds having cationic character, such as amine oxides, for example, alkyldimethylamine oxide or alkylaminoethyl dimethylamine oxide. The use of cetyltrimethyl ammonium salts is particularly advantageous.

### C. Amphoteric Surfactants

Amphoteric surfactants that can be advantageously used, include:

1. Acyl-/dialkylethyl endiamine, for example, sodium acyl amphoacetate, disodiumacyl amphodipropionate, disodium alkyl amphodiacetate, sodium acylamphohydroxy propylsulfonate, disodium acyl amphodiacetate, and sodium acyl amphopropionate;

2. N-alkylamino acids, for example, aminopropyl-alkylglutamide, alkylaminopropionic acid, sodium-alkylimidodipropionate and lauroamphocarboxyglycinate.

### D. Nonionische Surfactants

Nonionic surfactants, which can be advantageously used, include:

1. Alcohols;

2. Alkanolamides, such as MEA/DEA/MIPA cocoamides;

3. Amine oxides, such as cocoamidopropylamine oxide;

4. Esters, which result from the esterification of carboxylic acids with ethylene oxide, glycerin, sorbitan, or other alcohols;

5. Ethers, for example, ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE-ethers, and alkyl polyglycosides, such as lauryl glucoside, decyl glycoside and coco glycoside;

6. Sucrose esters, sucrose ethers;

7. Polyglycerol esters, diglycerol esters, monoglycerol esters; and

8. Methylglucose esters, esters of hydroxy acids.

It is also advantageous to use a combination of anionic and/or amphoteric surfactants with one or more nonionic surfactants.

In the preparations of the invention, the surface-active substance may be present in a concentration between 1 and 95 wt. % based on the total weight of the preparations.

The lipid phase of the cosmetic or dermatological emulsions according to the invention may advantageouly be selected from the following group of substances:

mineral oils, mineral waxes;

oils, such as triglycerides of capric or capryllic acids, furthermore natural oils, such as for example, castor oil;

fats, waxes, and other natural and synthetic lipoids, preferably esters of fatty acids with alcohols having a low carbon number, e.g., with isopropanol, propylene glycol or glycerol, or esters of fatty alcohols with alkanoic acids of a low carbon number or with fatty acids;

alkylbenzoates; and

silicone oils, such as dimethylpolysiloxanes, diethylpolysiloxanes, diphenylpolysiloxanes, as well as mixed forms thereof.

Advantageously, the oil phase of the emulsions of the present invention may further be selected from the groups of the esters of saturated and/or unsaturated, branched and/or unbranched alkane carboxylic acids having a chain length from 3 to 30 carbon atoms and saturated and/or unsaturated, branched and/or unbranched alkohols having a chain length from 3 to 30 carbon atoms; from the group of the esters of aromatic carboxylic acids and saturated and/or unsaturated, branched and/or unbranched alkohols having a chain length from 3 to 30 carbon atoms. Such ester oils may advantageously be selected from the group of isopropyl myristate, isopropylpalmitate, isopropyl stearate, isopropyl oleate, n-butyl stearate, n-hexyl laurate, n-decyl oleate, isooctyl stearate, isononyl stearate, isononyl isononanoate, 2-ethyl hexyl palmitate, 2-ethylhexyl laurate, 2-hexyldecyl stearate, 2-octyldodecyl palmitate, oleyl oleate, oley lerucate, erucyl oleate, erucyl erucate, as well as synthetic, semisynthetic, and natural mixtures of such esters, for example, jojoba oil.

Furthermore, the oil phase may advantageously be selected from the group of the branched and unbranched hydrocarbons and hydrocarbon waxes, the silicone oils, the dialkyl ethers, the group of the saturated or unsaturated, branched or unbranched alcohols, as well as the fatty acid triglycerides, namely the triglycerol esters of saturated and/or unsaturated, branched and/or unbranched alkane carboxylic acids having a chain length from 8 to 24, in particular 12 to 18 carbon atoms. The fatty acid triglycerides may adantageously be selected, for example, from the group of the synthetic, semisynthetic and natural oils, for example, olive oil, sunflower seed oil, soy oil, peanut oil, rape oil, almond oil, palm oil, coconut oil, palm kernel oil, and the like.

Any mixtures of such oil and wax components can also be used advantageously in the meaning of the present invention. In some instances, it may also be advantageous to use waxes, for example, cetyl palmitate, as the only lipid component of the oil phase.

Advantageously, the oil phase is selected from the group of 2-ethylhexylstearate, octyldodecanol, isotridecylisononanoate, isoeicosane, 2-ethylhexylcocoate, C₁₂₋₁₅-alkylbenzoate, triglyceride of the capryl-capric acid, dicaprylylether.

Especially advantageous are mixtures of C12-15 alkyl benzoate and 2-ethylhexyl isostearate, mixtures of C12-15 alkyl benzoate and isotridecyl isononanoate, as well as mixtures of C12-15 alkyl benzoate, 2-ethylhexyl-isostearate and isotridecyl isononanoate.

Of the hydrocarbons, paraffin oil, squalane and squalene are to be used with advantage in the meaning of the invention.

Advantageously, the oil phase can also advantageously include a content of cyclic or linear silicone oils or completely consist of such oils, although it is preferred to use an additional content of other oil phase components apart from the silicone oil or silicone oils. Such silicones or silicone oils may be present as monomers, which are normally characterized by structural elements, as follows:

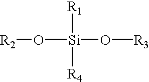

Linear silicones that are to be advantageously used according to the invention with a plurality of siloxyl units, are generally characterized by structural elements, as follows:

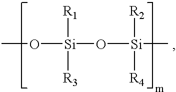

where the silicon atoms with the same or different alkyl radicals and/or aryl radicals may be substituted. They are here generally shown by the radicals R₁-R₄ (which means that the number of the different radicals is not necessarily limited to as many as 4), and m may assume values from 2 to 200,000.

Cyclical silicones that are to be advantageously used in accordance with the invention, are generally characterized by structural elements, as follows:

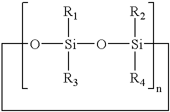

where the silicon atoms with the same or different alkyl radicals and/or aryl radicals, which are in general shown by the radicals R₁-R₄, may be substituted (which means that the number of the different radicals is not necessarily limited to as many as 4). n may assume values from 3/2 to 20. Broken values for n take into account that odd numbers of siloxyl groups may be present in the cyclic structure.

Advantageously, cyclomethicon (for example, decamethylcyclolpentasiloxane) is used as a silicone oil in accordance with the invention. However, other silicone oils can also be used advantageously in the meaning of the present invention, for example, undecamethylcyclotrisiloxane, polydimethylsiloxane, poly(methylphenylsiloxane), cetyldimethicon, and behenoxydimethicon.

Mixtures of cyclomethicon and isotridecyl isononanoate, as well as such of cyclomethicon and 2-ethyl hexyl isostearate are also advantageous. However, it is also advantageous to select silicone oils of a constitution similar to that of the above-described compounds, whose organic side chains are derivatized, for example, polyethoxylated and/or polypropoxylated. These include, for example, polysiloxanepolyalkyl-polyether-copolymers, such as the cetyl-dimethicon-copolyol, (cetyl-dimethiconcopolyol (and) polyglyceryl-4-isostearate (and) hexyl laurate).

Especially advantageous are also mixtures of cyclomethicon and isotridecyl isononanoate, of cyclomethicon and 2-ethyl hexyl isostearate.

Advantageously, the aqueous phase of the preparations according to the invention optionally include low-carbon alkohols, diols or polyols, as well as ethers thereof, preferably ethanol, isopropanol, propylene glycol, glycerol, ethylene glycol, ethylene glycol monoethyl or monobutyl ether, propylene glycol monomethyl, monoethyl, or monobutyl ether, diethylene glycolmonomethyl or monoethyl ether und analogous products, furthermore alcohols of a low carbon number, for example, ethanol isopropanol, 1,2-propandiol, glycerol as well as in particular, one or more thickeners, which can advantageously be selected from the group of silicon dioxide and aluminum silicate.

Preparations of the present invention that are present as emulsions, advantageously contain in particular one or more hydrocolloids. These hydrocolloids may advantageously be selected from the group of the gums, polysaccharides, cellulose derivatives, layer silicates, polyacrylates and/or other polymers.

Preparations of the invention that are present as hydrogels, contain one or more hydrocolloids. These hydrocolloids can advantageously be selected from the aforesaid group.

Gums include plant or tree saps, which cure in the air and form resins, or extracts from water plants. From this group, it is advantageous to select, in the meaning of the present invention, for example, gum arabicum, carob meal, tragacanth, Karaya, guar gum, pectin, gellan gum, carrageen, agar, algin, chondrus, xanthan gum.

Furthermore, it is advantageous to use derived gums, such as, for example, hydroxypropyl guar (Jaguar® HP 8).

Polysaccharides and derivatives thereof include, for example, hyaluronic acid, chitin, and chitosan, chondroitin sulfate, starch, and starch derivatives.

Cellulose derivatives include, for example, methylcellulose, carboxymethylcellulose, hydroxyethylcellulose, and hydroxypropylmethylcellulose.

Layer silicates include naturally occurring and synthetic clays, such as, for example, montmorillonite, bentonite, hectorite, laponite, magnesiumaluminium silicates, such as Veegum®. They may be used as such or in modified form, such as, for example, stearyl alkonium hectorite.

Furthermore, it is also advantageous to use silicic-acid gels.

Polyacrylates include, for example, Carbopol™ types from Goodrich (Carbopol™ 980, 981, 1382, 5984, 2984, EDT™ 2001 or Pemulen™ TR2).

Polymers include, for example, polyacrylamides (Seppigel™ 305), polyvinyl alcohols, PVP, PVP/VA copolymers, and polyglycols.

Preparations according to the invention, which are present as emulsions, include one more emulsifiers. Advantageously, these emulsifiers can be selected from the group of the nonionic, anionic, cationic, or amphoteric emulsifiers.

Nonionic emuslifiers include:

a) partial fatty acid esters and fatty acid esters of polyvalent alcohols and their ethoxylated derivatives (for example, glyceryl monostearates, sorbitan stearates, glyceryl stearyl citrates, sucrose stearates);

b) ethoxylated fatty alcohols and fatty acids;

c) ethoxylated fatty amines, fatty acid amides, fatty alkanolamides;

d) alkylphenolpolyglycolether (for example, Triton™ X).

Anionic emulsifiers include:

a) soaps (for example, sodium stearate);

b) fatty alcohol sulfates;

c) mono-, di- and trialkyl phosphoric acid esters and their ethoxylates.

Cationic emulsifiers include:

a) quaternary ammonium compounds with a long-chain aliphatic residue, for example, distearyl diammonium chlorides.

Among the amphoteric emsulsifiers are:

a) alkyl aminino alkane carboxylic acids;

b) betaines, sulfobetaines;

c) imidazolin derivatives.

Furthermore, there are naturally occurring emulsifiers, which include beeswax, wool wax, lecithin, and sterols.

O/W emulsifiers can advantageously be selected, for example, from the group of polyethoxylated or polypropoxylated or polyethoxylated and polypropoxylated products, for example:

fatty alcohol ethoxylates;

ethoxylated wool wax alcohols;

polyethylene glycol ethers of the general formula R—O—(—CH2—CH2—O—), —R′;

fatty acid ethoxylates of the general formula R—COO—(—CH₂—CH₂—O—)ₙ—H;

etherified fatty acid ethoxylates of the general formula R—COO—(—CH₂—CH₂—O)ₙ—R′;

esterified fatty acid ethoxylates of the general formula R—COO—(—CH₂—CH₂—O—)ₙ—C(O)—R′;

fatty acid esters of polyethylene glycolglycerin;

ethoxylated sorbitanester;

cholesterol ethoxylates;

ethoxylated triglycerides;

alkyl ether carboxylic acids of the general formula R—O—(—CH₂—CH₂—O—)ₙ CH₂—COON nd, where n is a number from 5 to 30;

fatty acid ester of polyoxyethylene sorbitol;

alkylether sulfates of the general formula R—O—(—CH₂—CH₂—O—ₙ—O₃—H;

fatty alcohol propoxylates of the general formula R—O—(—CH₂—CH(CH₃) —O—)ₙ—H;

polypropylene glycol ethers of the general formula R—O—(—CH₂—CH(CH₃)—O—)ₙR′;

propoxylated wool wax alcohols;

etherified fatty acid propoxylates R—COO—(—CH₂—CH(CH₃)—O—)ₙR′;

esterified fatty acid propoxylates of the general formula R—COO—(—CH₂—CH(CH₃)—O—)ₙC(O)—R′;

fatty acid propoxylates of the general formula R—COO—(—CH₂—CH(CH₃)—O—)ₙ—H;

fatty acid esters of polypropylene glycolglycerin;

propoxylated sorbitan esters;

cholesterol propoxylates;

propoxylated triglycerides;

alkyl ether carboxylic acids of the general formula R—O—(—CH₂—CH(CH₃)O—)ₙ—CH₂—COON;

alkylether sulfates or the parent acids of these sulfates of the general formula R—O—(—CH₂—CH(CH₃)—O—)ₙ—SO₃—H;

fatty alcohol ethoxylates/propoxylates of the general formula R—O-Xn-Y.—H;

polypropylene glycol ethers of the general formula R—O—Xₙ—Yₘ—R′;

etherified fatty acid propoxylates of the general formula R—COO—Xₙ—Yₘ R′;

fatty acid ethoxylates/propoxylates of the general formula R—COO—Xₙ—Yₘ—H,.

In accordance with the invention, it is especially advantageous to select the polyethoxylated or polypropoxylated or polyethoxylated and polypropoxylated O/W emulsifiers from the group of the substances with HLB values from 11 to 18, very advantageously with HLB values from 14.5 to 15.5, provided the O/W emulsifiers have saturated radicals R and R1. If the O/W emulsifiers include unsaturated radicals R and/or R1, or if isoalkyl derivatives are present, the preferred HLB value of such emulsifiers can also be lower or higher.

It is advantageous to select the fatty alcohol ethoxylates from the group of the ethoxylated stearyl alcohols, cetyl alcohols, cetyl stearyl alcohols (cetearyl alcohols). Especially preferred are:

polyethylene glycol(13)stearyl ether (steareth-13),

polyethylene glycol(14)stearyl ether (steareth-14),

polyethylene glycol(15)stearyl ether (steareth-15),

polyethylene glycol(16)stearyl ether (steareth-16),

polyethylene glycol(17)stearyl ether (steareth-17),

polyethylene glycol(18)stearyl ether (steareth-18),

polyethylene glycol(19)stearyl ether (steareth-19),

polyethylene glycol(20)stearyl ether (steareth-20),

polyethylene glycol(12)isostearyl ether (isosteareth-12),

polyethylene glycol(13)isostearyl ether (isosteareth-13),

polyethylene glycol(14)isostearyl ether (isosteareth-14),

polyethylene glycol(15)isostearyl ether (isosteareth-15),

polyethylene glycol(16)isostearyl ether (isosteareth-16),

polyethylene glycol(17)isostearyl ether (isosteareth-17),

polyethylene glycol(18)isostearyl ether (isosteareth-18),

polyethylene glycol(19)isostearyl ether (isosteareth-19),

polyethylene glycol(20)isostearyl ether (isosteareth-20);

Polyethylene glycol(13)cetyl ether (ceteth-13),

polyethylene glycol(14)cetyl ether (ceteth-14),

polyethylene glycol(15)cetyl ether (ceteth-15),

polyethylene glycol(16)cetyl ether (ceteth-16),

polyethylene glycol(17)cetyl ether (ceteth-17),

polyethylene glycol(18)cetyl ether (ceteth-18),

polyethylene glycol(19)cetyl ether (ceteth-19),

polyethylene glycol(20)cetyl ether (ceteth-20);

Polyethylene glycol (13) isocetyl ether (isoceteth-13),

polyethylene glycol (14) isocetyl ether (isoceteth-14),

polyethylene glycol (15) isocetyl ether (isoceteth-15),

polyethylene glycol (16) isocetyl ether (isoceteth-16),

polyethylene glycol (17) isocetyl ether (isoceteth-17),

polyethylene glycol (18) isocetyl ether (isoceteth-18),

polyethylene glycol(19)isocetyl ether (isoceteth-19),

polyethylene glycol(20)isocetyl ether (isoceteth-20);

Polyethylene glycol(12)oleyl ether (oleth-12),

polyethylene glycol(13)oleyl ether (oleth-13),

polyethylene glycol(14)oleyl ether (oleth-14),

polyethylene glycol(15)oleyl ether (oleth-15),

polyethylene glycol(12)lauryl ether (laureth-12),

polyethylene glycol(12)isolauryl ether (isolaureth-12);

Polyethylene glycol(13)cetylstearyl ether (ceteareth-13),

polyethylene glycol(14)cetylstearyl ether (ceteareth-14),

polyethylene glycol(15)cetylstearyl ether (ceteareth-15),

polyethylene glycol(16)cetylstearyl ether (ceteareth-16),

polyethylene glycol(17)cetylsteary lether (ceteareth-17),

polyethylene glycol(18)cetylstearyl ether (ceteareth-18),

polyethylene glycol(19)cetylstearyl ether (ceteareth-19),

polyethylene glycol(20)cetylstearyl ether (ceteareth-20).

It is also advantageous to select the fatty acid ethoxylates from the following group:

polyethylene glycol(20) stearate,

polyethylene glycol(21) stearate,

polyethylene glycol(22) stearate,

polyethylene glycol(23) stearate,

polyethylene glycol(24) stearate,

polyethylene glycol(25) stearate;

Polyethylene glycol(12) isostearate,

polyethylene glycol(13) isostearate,

polyethylene glycol(14) isostearate,

polyethylene glycol(15) isostearate,

polyethylene glycol(16) isostearate,

polyethylene glycol (17) isostearate

polyethylene glycol(18) isostearate,

polyethylene glycol(19) isostearate,

polyethylene glycol(20) isostearate,

polyethylene glycol(21) isostearate,

polyethylene glycol(22) isostearate,

polyethylene glycol(23) isostearate,

polyethylene glycol(24) isostearate,

polyethylene glycol(25) isostearate;

Polyethylene glycol(12) oleate,

polyethylene glycol(13) oleate,

polyethylene glycol(14) oleate,

polyethylene glycol(15) oleate,

polyethylene glycol(16) oleate,

polyethylene glycol(17) oleate,

polyethylene glycol(18) oleate,

polyethylene glycol(19) oleate,

polyethylene glycol(20) oleate.

Sodiumlaureth-11-carboxylate may advantageously be used as ethoxylated alkyl ether carboxylic acid or its salts.

Sodium laureth 1-4 sulfate may advantageously be used as an alkyl ether sulfate.

Polyethylene glycol(30)cholesteryl ether may be advantageously used as ethoxylated cholesterol derivative. Polyethylene glycol(25)soyasterol has also proven successful.

Polyethylene glycol(60) evening primrose glycerides may advantageously be used as ethoxylated triglycerides.

It is also advantageous to select the fatty acid esters of polyethylene glycol glycerol from the group of polyethylene glycol(20)glyceryl laurate,

polyethylene glycol(21)glyceryl laurate,

polyethylene glycol(22)glyceryl laurate,

polyethylene glycol(23)glyceryl laurate,

polyethylene glycol(6)glyceryl caprate/caprinate,

polyethylene glycol(20)glyceryl oleate,

polyethylene glycol(20)glyceryl isostearate,

polyethylene glycol(18)glyceryl oleate/cocoate.

It is likewise advantageous to select the sorbitan esters from the group of:

polyethylene glycol(20)sorbitan monolaurate,

polyethylene glycol(20)sorbitan monostearate,

polyethylene glycol(20)sorbitan monoisostearate,

polyethylene glycol(20)sorbitan monopalmitate,

polyethylene glycol(20)sorbitan monooleate.

It is possible to use as W/O emulsifiers: fatty alcohols having 8 to 30 carbon atoms, monoglycerol esters of saturated and/or unsaturated, branched and/or unbranched alkane carboxylic acids having a chain length from 8 to 24, in particular 12 to 18 carbon atoms, diglycerol esters of saturated and/or unsaturated, branched and/or unbranched alkane carboxylic acids having a chain length from 8 to 24, in particular 12 to 18 carbon atoms, monoglycerol ethers of saturated and/or unsaturated, branched and/or unbranched alcohols having a chain length from 8 to 24, in particular 12 to 18 carbon atoms, diglycerol ethers of saturated and/or unsaturated, branched and/or unbranched alcohols having a chain length from 8 to 24, in particular 12 to 18 carbon atoms, propylene glycol esters of saturated and/or unsaturated, branched and/or unbranched alkane carboxylic acids having a chain length from 8 to 24, in particular 12 to 18 carbon atoms, as well as sorbitan esters of saturated and/or unsaturated, branched and/or unbranched alkane carboxylic acids having a chain length from 8 to 24, in particular 12 to 18 carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2)stearyl ether (steareth-2), glyceryl monolaurate, glyceryl monocaprinate, glyceryl monocaprylate.

The following examples describe the invention in greater detail, without however limiting it. The numerical values relate to wt.-%, unless otherwise specified.

### EXAMPLE 1

| O/W Cream | Wt. % |
|---|---|
| Glyceryl stearate citrate | 4.00 |
| PEG-40-Stearate | 1.00 |
| Cetyl alcohol | 3.00 |
| Caprylic acid/capric acid triglycerides | 5.00 |
| Mineral oil | 5.00 |
| Lipoic acid | 0.20 |
| Tocopherol | 0.10 |
| Trisodium EDTA | 0.10 |
| Preservatives | q.s. |
| Carbomer | 3.00 |
| Sodium hydroxide solution 45% | q.s. |
| Glycerin | 5.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 2

| O/W Cream | Wt. % |
|---|---|
| Glyceryl sterate SE | 3.00 |
| Stearic acid | 1.00 |
| Cetyl alcohol | 2.00 |
| Caprylic acid/capric acid triglycerides | 3.00 |
| Dicaprylyl ether | 4.00 |
| Mineral oil | 2.00 |
| Lipoic acid | 0.50 |
| Preservatives | q.s. |
| Carbomer | 0.10 |
| Sodium hydroxide solution 45% | q.s. |
| Glycerin | 3.00 |
| Butylene glycol | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 3

| O/W Cream | Wt. % |
|---|---|
| Glyceryl stearate citrate | 2.00 |
| Stearyl alcohol | 5.00 |
| Caprylic acid/capric acid triglycerides | 4.00 |
| Octyl dodecanol | 2.00 |
| TiO₂ | 1.00 |
| 4-Methylbenzylidenecamphor | 1.00 |
| Butylmethoxydibenzolymethane | 0.50 |
| Lipoic acid | 0.10 |
| Biotin | 0.05 |
| Trisodium EDTA | 0.10 |
| Preservatives | q.s. |
| Carbomer | |
| Sodium hydroxide solution 45% | q.s. |
| Glycerin | 4.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 4

| O/W Cream | Wt. % |
|---|---|
| Polyglyceryl-3-methylglucose distearate | 3.00 |
| Cetyl alcohol | 3.00 |
| Caprylic acid/capric acid triglycerides | 3.00 |
| Dicaprylyl ether | 2.00 |
| Mineral oil | 3.00 |
| Lipoic acid | 1.00 |
| Trisodium EDTA | 0.10 |
| Preservatives | q.s. |
| Carbomer | 0.10 |
| Sodium hydroxide solution 45% | q.s. |
| Glycerin | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 5

| O/W-Cream | Wt. % |
|---|---|
| Glyceryl stearate citrate | 2.00 |
| Sorbitan stearate | 2.00 |
| Cetyl stearyl alcohol | 2.00 |
| Caprylic acid/capric acid triglycerides | 3.00 |
| Octyldodecanol | 2.00 |
| Dicaprylyl ether | 1.00 |
| Lipoic acid | 0.30 |
| Tocopherol | 0.20 |
| Preservatives | q.s. |
| Carbomer | 0.10 |
| Sodium hydroxide solution 45% | q.s. |
| Glycerin | 3.00 |
| Perfume | q.s. |
| Water | ad 100.00 |

The ingredients of the oil and water phases, respectively are combined, and the two phases are mixed together at 70-75° C., homogenized, and subsequently cooled to room temperature.

### EXAMPLE 6

| O/W-Cream | Wt. % |
|---|---|
| Glyceryl sterate 5 SE | 5.00 |
| Stearyl alcohol | 2.00 |
| Caprylic acid/capric acid triglycerides | 2.00 |
| Octyldodecanol | 2.00 |
| Dimethicon | 2.00 |
| TiO₂ | 2.00 |
| 4-Methylbenzylidene camphor | 1.00 |
| Butylmethoxydibenzolymethane | 0.50 |
| Lipoic acid | 0.20 |
| Preservatives | q.s. |
| Carbomer | 0.15 |
| Sodium hydroxide solution 45% | q.s. |
| Glycerin | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 7

| O/W-Cream | Wt. % |
|---|---|
| Glyceryl stearate citrate | 2.00 |
| Cetyl stearyl alcohol | 3.00 |
| C₁₂₋₁₅ alkyl benzoate | 2.00 |
| Octyldodecanol | 2.00 |
| Mineral oil | 4.00 |
| Lipoic acid | 0.70 |
| Preservatives | q.s. |
| Carbomer | 0.10 |
| Sodium hydroxide solution 45% | q.s. |
| Butylene glycol | 10.00 |
| Alcohol, denat. | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 8

| O/W-Cream | Wt. % |
|---|---|
| Glyceryl stearate citrate | 2.00 |
| Cetyl stearyl alcohol | 1.00 |
| C₁₂₋₁₅ alkyl benzoate | 3.00 |
| Mineral oil | 2.00 |
| Lipoic acid | 0.25 |
| Na₃HEDTA | 0.20 |
| Preservatives | q.s. |
| Xanthan gum | 0.20 |
| Sodium hydroxide solution 45% | q.s. |
| Glycerin | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 9

| O/W-Cream | Wt. % |
|---|---|
| Stearic acid | 2.50 |
| Cetyl alcohol | 3.00 |
| Octyldodecanol | 4.00 |
| Cyclomethicon | 0.50 |
| Lipoic acid | 2.00 |
| Preservatives | q.s. |
| Carbomer | 0.05 |
| Sodium hydroxide solution 45% | q.s. |
| Glycerin | 5.00 |
| Alcohol denat. | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 10

| O/W-Cream | Wt. % |
|---|---|
| Stearic acid | 3.50 |
| Cetyl alcohol | 4.50 |
| Cetyl stearyl alcohol | 0.50 |
| Octyldodecanol | 6.00 |
| Cyclomethicon | 2.00 |
| 4-Methylbenzylidene camphor | 1.00 |
| Butylmethoxydibenzolymethane | 0.50 |
| Lipoic acid | 0.40 |
| Tocopherol | 0.05 |
| Trisodium EDTA | 0.20 |
| Preservatives | q.s. |
| Carbomer | 0.05 |
| Sodium hydroxide solution 45% | q.s. |
| Glycerin | 3.00 |
| Butyleneglycol | |
| Alcohol, denat. | |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 11

| W/O Emulsion | Wt. % |
|---|---|
| Polyglyceryl-2-dipolyhydroxy stearate | 5.00 |
| Anisotriazine | 2.00 |
| Dioctylbutamidotriazone | 3.00 |
| Octocrylene | 7.00 |
| Dioctylbutamidotriazone | 1.00 |
| Bisimidazylate | 1.00 |
| Phenylbenzmidazolsulfonic acid | 0.50 |
| Zinc oxide | 3.00 |
| Dicaprylyl ether | 10.00 |
| Dicaprylyl carbonate | 5.00 |
| Cyclomethicon | 2.00 |
| PVP hexadecene copolymer | 0.50 |
| Glycerin | 3.00 |
| MgSO₄ | 1.00 |
| Vitamin E acetate | 0.50 |
| Liponic acid | 0.10 |
| Methylparaben | 0.50 |
| Phenoxyethanol | 0.50 |
| Ethanol | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 12

| W/O Emulsion | Wt. % |
|---|---|
| Cetyldimethiconcopolyol | 2.50 |
| Ethyl hexylmethoxy cinnamate | 8.00 |
| Anisotriazine | 2.50 |
| Dioctylbutamidotriazone | 1.00 |
| 4-Methylbenzylidene camphor | 2.00 |
| Octocrylene | 2.50 |
| Bisimidazylate | 2.00 |
| Titanium dioxide | 2.00 |
| Zinc oxide | 1.00 |
| Dimethicon | 4.00 |
| Cyclomethicon | 25.00 |
| Octoxyglycerin | 0.30 |
| Glycerin | 7.50 |
| Glycine soy | 1.00 |
| MgSO₄ | 0.50 |
| Lipoic acid | 0.60 |
| DMDM Hydantoin | 0.60 |
| Phenoxyethanol | 0.40 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 13

| W/O Emulsion | Wt. % |
|---|---|
| Cetyldimethiconcopolyol | 4.00 |
| Ethyl hexyl methoxy cinnamate | 5.00 |
| Anisotriazine | 2.00 |
| Butylmethoxydibenzoylmethane | 1.00 |
| Ethyl hexyltriazone | 4.00 |
| 4-Methylbenzylidene camphor | 4.00 |
| Dioctylbutamidotriazone | 2.00 |
| Phenylbenzmidazolsulfonic acid | 3.00 |
| Zinc oxide | 0.50 |
| C₁₂₋₁₅ alkyl benzoates | 9.00 |
| Butylene glycol dicaprylate/dicaprate | 8.00 |
| Dimethicon | 5.00 |
| PVP hexadecene copolymer | 0.50 |
| Glycerin | 7.50 |
| MgSO₄ | 0.50 |
| Lipoic acid | 1.00 |
| DMDM hydantoin | 0.20 |
| Methylparaben | 0.15 |
| Phenoxyethanol | 1.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 14

| W/O Emulsion | Wt. % |
|---|---|
| Polyglyceryl-2-dipolyhydroxy stearate | 4.50 |
| Ethyl hexylmethoxy cinnamate | 4.00 |
| Anisotriazine | 2.50 |
| Dioctylbutamidotriazone | 3.00 |
| 4-methylbenzylidene camphor | 2.00 |
| Octocrylene | 2.50 |
| Phenylbenzmidasolsulfonic acid | 2.00 |
| Titanium dioxide | 3.00 |
| Mineral oil | 8.00 |
| Dicaprylyl ether | 7.00 |
| Butylene glycol dicaprylate/dicaprate | 4.00 |
| Cyclomethicon | 2.00 |
| PVP hexadecene copolymer | 1.00 |
| Octoxyglycerin | 0.50 |
| Glycerin | 2.50 |
| MgCl₂ | 0.70 |
| Vitamin E acetate | 1.00 |
| Lipoic acid | 0.80 |
| Phenoxyethanol | 0.60 |
| Ethanol | 1.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 15

| W/O Emulsion | Wt. % |
|---|---|
| Polyglyceryl-2-dipolyhydroxy stearate | 4.00 |
| Wool wax alcohol | 0.50 |
| Isohexadecane | 1.00 |
| Myristylmyristate | 0.50 |
| Cera microcristallina + paraffinum liquidum | 1.00 |
| Butylmethoxydibenzoyl methane | 0.50 |
| 4-methylbenzylidene camphor | 1.00 |
| Butylene glycol dicaprylate/dicaprate | 4.00 |
| Glycerin | 5.00 |
| Vitamin E acetate | 0.50 |
| Lipoic acid | 0.20 |
| Na₃HEDTA | 0.20 |
| Methylparaben | q.s. |
| Phenoxyethanol | q.s. |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 16

| W/O Emulsion | Wt. % |
|---|---|
| Polyglyceryl-2-dipolyhydroxy stearate | 5.00 |
| Wool wax alcohol | 1.50 |
| Isohexadecane | 2.00 |
| Myristylmyristate | 1.50 |
| Cera Microcristallina + paraffinum liquidum | 2.00 |
| Butylmethoxydibenzoyl methane | 1.50 |
| 4-Methylbenzylidene camphor | 3.00 |
| Butylene glycol dicaprylate/dicaprate | 5.00 |
| Shea butter | 0.50 |
| Butyleneglycol | 6.00 |
| Octoxyglycerin | 3.00 |
| Vitamin E acetate | 1.00 |
| Lipoic acid | 0.25 |
| Na₃HEDTA | 0.20 |
| Methylparaben | q.s. |
| Phenoxyethanol | q.s. |
| Ethanol | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 17

| W/O Emulsion | Wt. % |
|---|---|
| PEG-30-dipolyhydroxy stearate | 5.00 |
| Butylmethoxydibenzoyl methane | 2.00 |
| Ethylhexyltriazone | 3.00 |
| Octocrylene | 4.00 |
| Bisimidazylate | 0.50 |
| Titanium dioxide | 1.50 |
| Zinc oxide | 2.00 |
| Mineral oil | 10.0 |
| Butylene glycol dicaprylate/dicaprate | 2.00 |
| Dicaprylyl carbonate | 6.00 |
| Dimethicon | 1.00 |
| Shea butter | 3.00 |
| Octoxyglycerin | 1.00 |
| Glycine Soja | 1.50 |
| MgCl₂ | 1.00 |
| Vitamin E acetate | 0.25 |
| Lipoic acid | 1.50 |
| DMDM hydantoin | 0.40 |
| Methylparaben | 0.25 |
| Ethanol | 1.50 |
| Perfume | |
| Water | ad 100.00 |

The ingredients of the oil and water phases, respectively, are combined, and the two phases are mixed together at 70-75° C., homogenized, and subsequently cooled to room temperature.

### EXAMPLE 18

| Hydrodispersion | Wt. % |
|---|---|
| Polyoxyethylene (20) cetyl stearyl ether | 1.00 |
| Acrylate/C10-30 alkylacrylate crosspolymer | 0.50 |
| Butylmethoxydibenoyl methane | 1.00 |
| Ethylhexyltriazone | 4.00 |
| 4-methylbenzylidene camphor | 4.00 |
| Dioctylbutamidotriazone | 1.00 |
| Bisimidazylate | 1.00 |
| Phenylbenzmidazolsulfonic acid | 0.50 |
| Titanium oxide | 0.50 |
| Zinc oxide | 0.50 |
| C₁₂₋₁₅alkylbenzoates | 2.00 |
| Butylene glycol dicaprylate/dicaprate | 4.00 |
| Phenyltrimethicons | 2.00 |
| PVP hexadecene copolymer | 0.50 |
| Glycerin | 3.00 |
| Vitamin E acetate | 0.50 |
| Lipoic acid | 0.15 |
| Koncyl-L ® | q.s. |
| Methylparaben | q.s. |
| Phenoxyethanol | q.s. |
| Ethanol | 3.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 19

| Hydrodispersion | Wt. % |
|---|---|
| Sodiumcarbomer | 0.20 |
| Xanthan gum | 0.30 |
| Anisotriazine | 1.50 |
| Dioctylbutamidotriazone | 2.00 |
| 4-methylbenzylidene camphor | 4.00 |
| Octocrylene | 4.00 |
| Zinc oxide | 1.00 |
| C₁₂₋₁₅ alkyl benzoates | 2.50 |
| Dicaprylyl ether | 4.00 |
| Dicaprylyl carbonate | 2.00 |
| Dimethicon | 0.50 |
| Shea butter | 2.00 |
| Glycerin | 7.50 |
| Lipoic acid | 0.60 |
| DMDM hydantoin | 0.60 |
| Koncyl-L ® | q.s. |
| Methylparaben | q.s. |
| Phenoxyethanol | q.s. |
| Ethanol | 2.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 20

| Hydrodispersion | Wt. % |
|---|---|
| Cetylalcohol | 1.00 |
| Acrylate/C₁₀₋₃₀alkylacrylate crosspolymer | 0.40 |
| Xanthan gum | 0.15 |
| Butylmethoxydibenzoyl methane | 2.00 |
| Ethyl hexyltriazone | 3.00 |
| Octocrylene | 4.00 |
| Bisimidazylate | 0.50 |
| Titanium dioxide | 2.00 |
| Zinc oxide | 3.50 |
| Butylene glycol dicaprylate/dicaprate | 2.00 |
| Dicaprylyl carbonate | 6.00 |
| Dimethicon | 1.00 |
| Octoxyglycerin | 1.00 |
| Glycine soy | 1.50 |
| Vitamin E acetate | 0.25 |
| Lipoic acid | 1.50 |
| DMDM hydantoin | 0.40 |
| Koncyl-L ® | q.s. |
| Methylparaben | q.s. |
| Phenoxyethanol | q.s. |
| Ethanol | 1.50 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 21

| Hydrodispersion | Wt. % |
|---|---|
| Polyoxyethylene (20) cetyl stearyl ether | 0.50 |
| Sodiumcarbomer | 0.30 |
| Acrylate/C₁₀₋₃₀alkylacrylate crosspolymer | 0.10 |
| Ethyl hexylmethoxy cinnamate | 5.00 |
| Anisotriazine | 2.00 |
| Dioctylbutamidotriazone | 2.00 |
| Ethyl hexyltriazone | 4.00 |
| Dioctylbutamidotriazone | 2.00 |
| Phenylbenzmidazol sulfonic acid | 3.00 |
| Titanium dioxide | 3.00 |
| Butylene glycol dicaprylate/dicaprate | 6.00 |
| Phenyltrimethicon | 0.50 |
| PVP Hexadecene copolymer | 0.50 |
| Glycerin | 7.50 |
| Lipoic acid | 1.00 |
| DMDM hydantoin | 0.20 |
| Koncyl-L ® | q.s. |
| Methylparaben | q.s. |
| Phenoxyethanol | q.s. |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 22

| Hydrodispersion | Wt. % |
|---|---|
| Acrylate/C₁₀₋₃₀alkylacrylate crosspolymer | 0.10 |
| Xanthan gum | 0.50 |
| Ethyl hexylmethoxy cinnamate | 8.00 |
| Anisotriazine | 2.50 |
| Dioctylbutamidotriazone | 1.00 |
| 4-methylbenzylidene camphor | 2.00 |
| Octocrylene | 2.50 |
| Bisimidazylate | 2.00 |
| Titanium dioxide | 1.00 |
| Zinc oxide | 2.00 |
| Phenyltrimethicons | 2.00 |
| PVP hexadecene copolymer | 1.00 |
| Octoxyglycerin | 0.50 |
| Glycerin | 2.50 |
| Vitamin E acetate | 1.00 |
| Lipoic acid | 0.80 |
| Koncyl-L ® | q.s. |
| Methylparaben | q.s. |
| Phenoxyethanol | q.s. |
| Ethanol | 1.00 |
| Perfume | q.s. |
| Water | ad 100.00 |

The ingredients of the oil and water phases, respectively, are combined, and the two phases are mixed together at 70-75° C., homogenized, and subsequently cooled to room temperature.

### EXAMPLE 23

| Gel cream | Wt. % |
|---|---|
| Acrylate/C₁₀₋₃₀alkylacrylate crosspolymer | 0.40 |
| Carbomer | 0.20 |
| Xanthan gum | 0.10 |
| Cetyl stearyl alcohol | 3.00 |
| C₁₂₋₁₅ alkylbenzoates | 4.00 |
| Caprylic acid/capric acid triglycerides | 3.00 |
| Cyclomethicon | 5.00 |
| Dimethicons | 1.00 |
| Lipoic acid | 0.20 |
| Glycerin | 3.00 |
| Sodium hydroxide | q.s. |
| Preservation | q.s. |
| Perfume | q.s. |
| Water, demineralized | ad 100.00 |
| pH-value adjusted to 6.0 | |

The ingredients of the oil and water phases, respectively, are combined, and the two phases are mixed together at 70-75° C., homogenized, and subsequently cooled to room temperature.

### EXAMPLE 24

| W/O-Cream | Wt. % |
|---|---|
| Lameform ® TGI | 3.50 |
| Glycerin | 3.00 |
| Dehymuls ® PGPH | 3.50 |
| Lipoic acid | 0.50 |
| Preservatives | q.s. |
| Perfume | q.s. |
| Magnesium sulfate | 0.6 |
| Isopropyl stearate | 2.0 |
| Dicaprylyl ether | 8.0 |
| Cetyl stearyl isononanoate | 6.0 |
| Wasser, demin. | ad 100.00 |

The ingredients of the oil and water phases, respectively, are combined, and the two phases are mixed together at 70-75° C., homogenized, and subsequently cooled to room temperature.

### EXAMPLE 25

| W/O/W-Cream | Wt. -% |
|---|---|
| Glyceryl stearate | 3.00 |
| PEG-1 00-stearate | 0.75 |
| Behenyl alcohol | 2.00 |
| Caprylic acid/capric acid triglycerides | 8.00 |
| Octyldodecanol | 5.00 |
| C₁₂₋₁₅ alkyl benzoates | 3.00 |
| Lipoic acid | 1.00 |
| MgSO₄ | 0.80 |
| EDTA | 0.10 |
| Preservation | q.s. |
| Perfume | q.s. |
| Water, demineralized | ad 100.00 |
| pH-value adjusted to 6.0 | |

The ingredients of the oil phase are combined and homogenized. Subsequently, they are combined with the water phase and heated to a temperature of 80-85° C. (i.e., brought into the phase inversion temperature range of the system). Subsequently, they are cooled to room temperature (i.e., removed from the phase inversion temperature range of the system).

1. Use of α-lipoic acid for producing cosmetic or dermatological preparations for regenerating stressed skin, in particular aged skin.

2. Use of claim 1, characterized in that the preparations contain 0.001 to 10 wt. % of lipoic acid based the total weight of the preparations.
